(19) United States
(12) Patent Application Publication
(10) Pub. No.: US 2026/0215692 A1
(43) Pub. Date: Jul. 30, 2026

(54) Arrangement for determining a pulse wave transit time of an object, computer-implemented method and computer program product

(71) Applicant: BIOLAB Innovation GmbH, Berlin (DE)

(72) Inventors: Márton DANÓCZY, Berlin (DE); Giovanna DEL VECCHIO, Berlin (DE); Serop BAGHDADLIAN, Berlin (DE)

(73) Assignee: BIOLAB Innovation GmbH, Berlin (DE)

(21) Appl. No.: 19/146,044

(22) PCT Filed: Jan. 15, 2024
(86) PCT No.: PCT/EP2024/050805

§ 371 Date: Jul. 7, 2025

(30) Foreign Application Priority Data
Jan. 17, 2023 (EP) 23152098.2

(51) Int. Cl. A61B 5/021; A61B 5/024

(52) U.S. Cl. CPC A61B 5/02125; A61B 5/02416

(57) ABSTRACT

Arrangement for determining a pulse wave transit time of an object, including a light source configured to irradiate a first surface of the object with light, and a light sensor configured to detect light emanating from the first surface of the object as a light signal, and a computer device configured to calculate a photoplethysmogram on the basis of the light signal. A motion sensor is configured to detect a movement of a second surface of the object as a motion signal. The computer device is configured to provide a seismocardiogram on the basis of the motion signal, and to synchronize the photoplethysmogram and the seismocardiogram, and to determine local extreme values over the course of time of the photoplethysmogram and the seismocardiogram, and taking into consideration local extreme values, to determine the pulse wave transit time between areas of the object associated with the first and second surfaces.

### CROSS-REFERENCE TO RELATED APPLICATIONS

This application is the United States National Phase under 35 U.S.C. § 371 of PCT International Patent Application No. PCT/EP2024/050805, filed on Jan. 15, 2024, which claims the benefit of European Patent Application No. 23152098.2, filed on Jan. 17, 2023, the disclosures of which are hereby incorporated by reference herein in their entireties.

### TECHNICAL FIELD

The present invention relates to an arrangement for determining a pulse wave transit time of an object according to the preamble of claim 1, as well as a computer-implemented method according to the preamble of claim 11 and a computer program product according to claim 15.

### BACKGROUND

Recording the biological parameters of a person, such as for example the pulse, blood pressure, respiratory rate, oxygen saturation, pulse wave variability and blood sugar, is becoming increasingly widespread in everyday life and is no longer used purely for medical purposes. In the past, they were only used for a so-called patient chart, for example in a hospital or in competitive sports. They were used for monitoring patients before or after treatment in order to accurately document their state of health and record any improvement or deterioration. In competitive sports, recorded biological parameters are used to document the performance of an athlete and determine the success of training. It can be determined, for example, whether the athlete's training or diet needs to be changed.

Biological parameters have also become of interest and importance for home diagnosis and recreational sports. Thus, for example, patients are no longer necessarily hospitalized but are often only treated as outpatients, with follow-up care taking place at home. For example, patients may need to record their own pulse after treatment. A device is often used for this which has a chest strap with sensors and a recording device. The sensors of the chest strap record the pulse beats directly on the patient's chest. The data is then transmitted to the recording device. The recording device can store the data over several hours to days, for example for a long-term-cardiogram. The data is then read out and analyzed by medical staff for example.

However, the device is uncomfortable for patients to wear, as the chest strap has to be worn without interruption for a long-term cardiogram for example. This restricts the patient's mobility and is also inconvenient for daily washing. Furthermore, the patient also needs to carry the recording device around with them at all times. The same applies to long-term blood pressure measurements, where a blood pressure cuff needs to be worn by a patient e.g. for over 24 hours, with the blood pressure cuff being pressurized every 15 minutes for example.

From publication WO 2014/072461 A1 it is known to illuminate an area of a person's skin using a smartphone and to use an internal camera to record image data of the blood flow through blood vessels that run close to the surface of the skin. A pulse wave transit time is estimated from the image data by using a photoplethysmogram. From the pulse wave transit time or the pulse wave velocity it is possible to infer the current blood pressure.

Based on earlier approaches for determining pulse wave transit times, the objective of the invention is to provide an arrangement by which a pulse wave transit time can be determined comparatively simply, accurately and inexpensively.

The present disclosure is directed toward overcoming one or more of the above-mentioned problems, though not necessarily limited to embodiments that do.

### SUMMARY

The invention solves this problem by means of an arrangement according to claim 1.

An arrangement comprises e.g. a smartphone or a tablet. In this case, many or all of the components essential to the invention are integrated into a common housing. Alternatively, the assemblies can be accommodated in several housings and communicate with one another by cable or wirelessly by radio (e.g. Bluetooth, 3G, 4G, 5G, etc.). Time synchronization is important here and can be achieved wirelessly, e.g. by means of the “network time protocol” (available for example from the website https://datatracker.ietf.org/doc/html/rfc5905).

For example, the light sensor and light source can be combined in one housing, while the motion sensor is provided in a separate housing. The computer device and/or a display device, such as a screen, can be provided separately or in one of the two aforementioned housings. Accordingly, individual or all components that are not used for direct measurement on the object (such as the light source, light sensor, motion sensor) can be provided remotely on a server device that is connected to the aforementioned components via a data communication link, e.g. via the internet. The components that are not used for direct measurement on the object (i.e. the computer device and any software components) can also be provided in a cloud. A cloud is e.g. a system with a plurality of data processor devices and data storage devices which are connected via an internal network in a data center or in the case of a plurality of data centers e.g. via the internet, wherein by means of a special software architecture calculation tasks can be scaled easily and quickly beyond the capacity of a single data processor or data storage device.

The arrangement can alternatively also be formed by a smartwatch, which comprises a motion sensor, and data glasses for “augmented reality”, which have a camera and a flash. A smartwatch or data glasses, which combine a light source, a camera and a motion sensor, can also be used.

An object within the meaning of the invention is e.g. a human or animal body. A first surface is for example an area of skin in the periphery of the blood circulation, e.g. a fingertip of a human user. A second surface is e.g. an area of skin in the vicinity of the heart (for example closer than 25 cm, preferably closer than 10 cm apart). The second surface is preferably e.g. the skin surface of the human sternum. The area of the object associated with the first surface is e.g. a part of the vascular system located under the skin of the fingertip. The area of the object assigned to the second surface of the object is for example a human heart.

A pulse wave transit time within the meaning of the invention is e.g. the period of time that elapses between an opening of the aortic valve of the heart during a heartbeat and the arrival of the positive beat (in the sense of an increase in blood pressure) in the fingertip. The pulse wave velocity can be calculated from the pulse wave transit time, taking into consideration the spatial distance between the heart and the fingertip. The formula is:

$$pulse wave velocity = distance / pulse wave transit time .$$

The distance can be measured or estimated directly on the body by means of a measuring tape or the like.

In order to measure the arterial vessel length, for example, a method can be used as described in the publication “A Pulse Wave Velocity Based Method to Assess the Mean Arterial Blood Pressure Limits of Autoregulation in Peripheral Arteries”, by A. Tripathi et al., in the Journal Front Physiol., 2017. Tripathi et al. suggest on page 2, column 2, last sentence to page 3, left column para, “Calculations” to measure the span D_{w} as the distance of the left index finger to the right index finger with the arms spread 90 degrees apart in the lateral position. The pulse wave transit time is measured for up to the right index finger and up to the left index finger. The results in:

$$pulse wave {velocity}_{left} = {D}_{w} / ( 2 * pulse wave transit {time}_{left} )$$

$$pulse wave {velocity}_{right} = {D}_{w} / ( 2 * pulse wave transit {time}_{right} ) .$$

The values for left and right can be averaged to determine a unit value for the pulse wave velocity.

Furthermore, half the span can also be measured in the same way, in each case from the sternal notch to the left or right index finger. These measurement values can be used to determine the pulse wave velocity in the same way.

This can be further simplified by measuring the so-called “demispan” as the distance between the suprasternal notch and the root of the middle finger, according to Tan et al. (“The Arm Span to Height Relationship and Its Health Implications”, V. R. Preedy (ed.), Handbook of Anthropometry: Physical Measures, 2012), page 744. Empirical equations are proposed to determine “demispan” on a gender-specific basis:

$$Women : body height ( cm ) = 1.35 \times demispan ( cm ) + 60.1 cm$$

$$Men : body height ( cm ) = 1.4 \times demispan ( cm ) + 57.8 cm .$$

Thus, for example, the gender and body height can be simply queried from an app on a smartphone for the method according to the invention in order to estimate a span value and determine the pulse wave velocity by using this span value. This is a preferred approach according to the invention.

As an alternative, a simplified method based on body height can be used. The distance d is taken to be approximately half the height h:

$$d = h / 2 ,$$

where d corresponds to the distance between the so-called sternal notch and the tip of the middle finger with the arm extended at a 90° angle to the body. Possible sources of error when calculating the pulse wave velocity according to this approach need to be taken into account:
d does not correspond to the arterial path, resulting in systematic deviations.
Although this approach is based on the well-established concept that the ratio between the body height and arm span (2d) is approximately 1 (such as for example in the “Vitruvian Man” by Da Vinci), gender and ethnicity are known factors that influence this ratio.
The longest finger (middle finger) is used as a reference for the end of the measured distance. In contrast, the index finger is generally used to determine the pulse wave velocity according to the invention. This may result in a further small deviation due to the system, but this may be negligible.

For example, a measurement of the pulse wave transit time can be averaged over a predetermined number of heartbeats. For example, more than 10, preferably more than 50, even more preferably 80-100 heartbeats of a person can be recorded (this corresponds in absolute terms to a measurement duration of about one minute) and the pulse wave transit times determined in each case are averaged in order to subsequently calculate the pulse wave velocity. A typical range for a pulse wave velocity of a person is e.g. between 5 m/s to 10 m/s, with a measurement precision of approx. +/−10% for several measurements of the same person.

A longer pulse wave transit time or a low pulse wave velocity is an indicator of comparatively lower arterial vascular stiffness and lower blood pressure, i.e. more elastic blood vessels. As a rule, healthy younger users therefore have a longer pulse wave transit times on average than older users. The duration and velocity of the pulse wave can therefore be used to draw conclusions about the condition of the vascular wall. Advantageously, it is possible for a user to use the measurement results to diagnose an arteriosclerotic change in the vessels at a very early stage themselves or in cooperation with a physician and to prevent the progression of arteriosclerosis with a suitable change in lifestyle (e.g. low-fat and low-sodium diet, physical activity).

The method according to the invention can thus be used to draw conclusions about the cardiovascular health of a user, wherein the user can carry out regular mobile health checks without the need to visit a physician due to the ease of using the existing smartphone.

For example, a pulse wave transit time measurement can be carried out daily, weekly or monthly for a user of the arrangement and the pulse wave velocity can be determined. This allows a long-term trend in the pulse wave velocity to be determined. For example, if a person has a pulse wave transit time of 7.3 m/s at the start of the long-term measurement and this value increases over time, this may indicate a deteriorating vascular condition. In this case, the user can be shown a recommendation to visit a physician to check their state of health. The device is therefore also ideal for use in the field of telemedicine, particularly if the measured values are automatically transmitted to a physician e.g. via the internet.

Of course, this requires the user's prior consent. In the first step, a user can identify themselves, for example by collecting biometric data. This may for example involve recognizing a fingerprint with a fingerprint scanner or a suitable screen. Alternatively or additionally, facial recognition can be carried out using a camera, preferably in conjunction with a 3D face scan. This type of functionality is known for example from Apple products with the brand name “Face ID”.

Once the user has been identified, a comparison can be made with previously recorded biometric data that is linked to the name and/or other personal information such as data of birth and home address. For example, in the case of an Apple smartphone the data record determined during the creation of a so-called “Apple ID” enables payment on the internet and ultimately unique identification of the user.

For example, users can be notified by a display on a screen or an audio output that a data transfer to the physician is recommended and that users should identify themselves with their face if they agree. Identification by fingerprint, voice or password can also be used advantageously. If consent is given, consent information is also transmitted electronically to the physician as a data message together with the measured pulse wave velocity and/or pulse wave transit time values. Furthermore, the data message can also include an authentication confirmation of the user's identity.

In addition to disease-related changes in the pulse wave transit time and/or pulse wave velocity, lifestyle dependent changes in the vascular condition, e.g. as a result of a long-term diet or a long-term sports program, can also be visualized.

It is particularly useful to draw conclusions about the current blood pressure based on the detected pulse wave transit time and/or pulse wave velocity. High blood pressure is a significant risk factor for heart attacks or strokes and is therefore usually lowered by medication as soon as it is recognized by the physician.

Blood pressure is one of the medical standards for assessing the cardiovascular situation at rest and during physical exertion. The physiological limits at rest and under stress are described in detail and established in guidelines. However, it is currently not possible to continuously determine blood pressure under stress, as the blood pressure can only be determined at set time points using a blood pressure cuff. Continuous measurement is only made possible by the invention. According to the invention, the blood pressure is determined from the velocity of the pulse wave or the pulse wave transit time, a short transmit time from the heart to the finger indicates high blood pressure, as the vessels are comparatively stiffer. Preferably, when using the invention for measuring blood pressure, calibration is carried out at rest and under stress. Then the blood pressure can be measured continuously.

In a simple approach, it is possible to use calibration measurements of a large number of people with known blood pressure (blood pressure is measured e.g. immediately after the pulse wave transit time measurement by means of a blood pressure cuff) to create a calibration curve which allows a determined pulse wave transit time and/or pulse wave velocity to be converted into an instantaneous blood pressure. Both the systolic and diastolic blood pressure of the user can be estimated.

If elevated blood pressure and/or a tendency towards high blood pressure worsening over time is recognized, a visit to the physician may be recommended.

To determine a blood pressure value on the basis of the pulse wave transit time, Mukkamala et al. propose various mathematical approaches for conversion in “Towards Ubiquitous Blood Pressure Monitoring via Pulse Transit Time: Theory and Practice”, IEEE Trans Biomed Eng. 2015 August; 62 (8): 1879-1901., page 19. These approaches can advantageously be used in the context of the present invention for calculating and outputting or displaying an instantaneous blood pressure value for a user based on the pulse wave transit time measurement according to the invention.

The arrangement can comprise a display means, for example for displaying the pulse wave transit time determined according to the invention or a pulse wave velocity calculated therefrom. This measurement value can be set in relation to pulse wave transit times or pulse wave velocities which are expected on average for persons of the same sex and age. The display means can for example include a screen, a digital display for letter and numbers (similar to radio alarm clocks) or a touchscreen.

The light source comprises for example a light-emitting diode (LED) that emits white light in the visible wavelength range between 380 nm and 690 nm wavelength. For example, a permanently switched on LED can be used for the flash of a smartphone or tablet.

Alternatively, infrared light with a wavelength above 690 nm to 3 μm can also be used in combination with red visible light (640 nm to 690 nm wavelength), as is common with pulse oximeters, to determine the oxygen saturation of the blood based on the different absorption properties of light for oxidized and non-oxidized hemoglobin. In principle, the wavelength range used can be adjusted to an absorption maximum of oxidized and/or non-oxidized hemoglobin. Traditional pulse oximeters use two light sources for this purpose: a red light-emitting diode for a wavelength of 660 nm and an infrared light-emitting diode for a wavelength of 940 nm.

The light sensor is preferably configured for detecting the light emitted by the light source, i.e. it can detect the corresponding wavelength ranges by means of one or more photodiodes or the like. Preferably, the light sensor is configured for visible light. It can be in the form of a smartphone camera for example. The light signal is preferably a digital signal. If a simple photodiode is used, a time curve of the light intensity can be recorded directly. This analogue signal (usually a voltage curve over time) can be converted into a digital light signal using an analogue-to-digital converter for example.

For example, by using a smartphone or a tablet it is possible to take a measurement in such a way that a reclining user places the device on their chest, by positioning the device e.g. vertically on an area of skin over the sternum or over the heart in the 4th intercostal space, and placing a finger on the camera and the flash of the device. According to the invention, it is possible to determine which position is more suitable either on the sternum or over the heart at the 4th intercostal space by carrying out a series of measurements for a user in both positions e.g. lasting one minute. This produces two seismocardiograms, wherein the preferred position is the one in which the seismocardiogram has the highest amplitudes (i.e. the vibrations in conjunction with the heart activity are damped the least by the tissue between the skin surface and the heart).

The motion sensor of such a device is generally in the form of an accelerometer, which can detect accelerations in three spatial dimensions. According to the invention, the movement pulse resulting from the closing of the ventricular value can be measured most strongly approximately perpendicular to the sternum, so that in a preferred simplified method, e.g. when the device is positioned vertically “upright” on the chest of a reclining user, only the spatial dimension with the greatest measured values needs to be further evaluated and the other two spatial dimensions or the data recorded for them can be discarded. The dimension with the largest measured values is e.g. the dimension in which the vector g of the acceleration due to gravity occurs. The motion sensor should preferably be operated at the highest possible time resolution, which is typically at least 400 Hz. Preferably, the motion sensor can provide data at at least 100 Hz. If unexpectedly high accelerations are detected in one of the spatial dimensions during the measurement, it can be assumed that the object is moving and the measurement can be abandoned. A threshold value for a maximum permissible acceleration can be determined. For example, a threshold value for the acceleration of half the acceleration due to gravity (0.5 g, i.e. approx. 5 m/s²) can be used.

The aforementioned publication WO 2014/072461 A1 does disclose the use of a motion sensor, however the latter is only used for recognizing a user's sporting activity in a series of measurements. A seismocardiogram is not generated and analyzed.

The computer device can comprise a microprocessor (CPU), a graphics processor (GPU) or an ASIC for example. Furthermore, a data storage device can be used such as a RAM or a flash memory or a solid-state disc (SSD) for the intermediate storage of data. For example, the data processor of a smartphone or a tablet can be used for the computer device.

A seismocardiogram (SCG) is e.g. a one-dimensional time curve of vibrations that can be measured on a surface. A photoplethysmogram (PPG) is e.g. a one-dimensional time curve of color change values of a skin surface, that occur when an incoming pulse wave briefly dilates the arterial vessels under the skin so that more blood and thus more red blood cells are present.

Time synchronization means that the photoplethysmogram and the seismocardiogram are related to one another in such a way that both series of measurements begin at the same point in time.

Local extreme values are maximum and/or minimum values which occur periodically over the measurement period (for each heartbeat).

In a preferred embodiment of the arrangement according to the invention, the light sensor comprises a camera which is configured to capture a timeline of individual images with a predetermined frame rate and predetermined image resolution. The camera is preferably switched to automatic mode, wherein all settings such as e.g. autofocus are set as constant for the measurement period (typically at least one minute) in order to avoid artefacts in the measurement. The flash is activated permanently to illuminate the object as a light source.

A color camera or a black and white camera can be used. Preferably, a color camera is used which is referred to in the following.

If for example a middle color in the red color range is detected for more than one second, it can be assumed that an area of skin has been placed on the sensor and the measurement can begin.

The camera is preferably captured with the maximum possible frame rate and minimum possible triggering. Typically, a modern smartphone can provide a frame rate of 60 Hz, wherein at least 30 Hz are preferred according to the invention.

In a further preferred embodiment of the arrangement according to the invention the computer device is configured to convert the individual images into a one-dimensional time curve, wherein for each individual image color values of pixels are added up, and to provide these added up color values in the time curve as a photoplethysmogram.

According to the invention, at least one one-dimensional progression is evaluated. Alternatively however, a multi-dimensional progression can also be used, in that e.g. two or three of the color channels described in more detail below are taken into account for the evaluation. Thus results in a two-dimensional or three-dimensional progression.

Typically a color camera records three color values for each pixel of an individual image, namely red, green and blue (so-called RGB values). According to the invention, all red values in a single image can be averaged to obtain an average red value Rp of the individual image. In the same way, an average blue value Bp and an average green value G_{D} can be determined. A color value F for an individual image can then be obtained by adding up the three average values and setting the result to negative (this is simply a common convention in the scientific literature):

$$F = - ( {R}_{D} + {G}_{D} + {B}_{D} ) .$$

The photoplethysmogram (PPG) is then obtained as a timeline of the F values of the individual images. In this way, a comparatively meaningful PPG is achieved.

In contrast to the use of three colours, a similar result can be achieved with a black and white camera by averaging the grey values. If the camera provides three color values, only the red value can be considered for each image and the other color values remain disregarded.

Alternatively or additionally, in some types of camera the green value can also be evaluated and the other color channels disregarded. This is useful if the flash is so strong that the red channel is saturated (R=255) and the finger to be measured appears orange or yellow (i.e. R+G). In such a case, only the change in the green color channel is significant for the photoplethysmogram.

Typically all pixels of an individual image are used. In a further development of this approach, a digital template can also be used for the area of the object captured by the camera (for example a mask with value 1 in a circular section and otherwise a value of 0 is multiplied by the individual image), for example to evaluate only a circular image section in the center of the image with regard to the color values.

When detecting the PPG, upper and lower limit values can be set for each color in the individual images in order to quickly identify and cancel incorrect measurements. For example, measured values that are too low could indicate that a finger has not been positioned correctly on the light sensor and ambient light is interfering with the measurement. The limit values can be recalculated for each image capture in order to compensate for different recording conditions. For example, the amplitude of each local maximum value can be determined in the photoplethysmogram in order to determine the median of the local maximal values. Subsequently, all local maximum values for further evaluation can be disregarded which are twice as large as the median.

In a further preferred embodiment of the arrangement according to the invention the computer device is configured to convert the motion signal into a one-dimensional time curve, wherein acceleration values are recorded in several spatial dimensions, wherein for the spatial dimension with the largest acceleration values, the respective acceleration value is take into account at each measurement time point for providing the seismocardiogram.

In an alternative embodiment of the arrangement according to the invention the computer device is configured to convert the motion signal into a multidimensional time curve, wherein acceleration values are recorded in several spatial dimensions. This has the advantage that e.g. a two-dimensional or three-dimensional time curve contains additional information about for example a change in the tilting angle of the smartphone positioned on the chest of a user which can be analyzed advantageously. For example, incorrect measurements can be recognized more effectively in this way.

In a further preferred embodiment of the arrangement according to the invention the computer device is configured to carry out resampling of the photoplethysmogram and seismocardiogram to a common time resolution. The resampling is carried out before the time synchronization of the photoplethysmogram and seismocardiogram. Preferably, a common time resolution (i.e. a common sampling rate) of more than 500 Hz, even more preferably of at least 1000 Hz is used. This is advantageous as otherwise for example the motion signal could have a sampling rate four times higher than the light signal. In general the resampling is therefore an upsampling of the two signals.

In a further preferred embodiment of the arrangement according to the invention, the computer device is configured to use Hermite interpolation for resampling. This is an advantage, because this method produces a comparatively exact result. A so-called sub-sample precision, i.e. a better sampling rate than the original signal, can be achieved, wherein in particular effects caused by irregular sampling functions (e.g. frame rate during the measurement period no maintained exactly) are minimized.

The basic principle of Hermite interpolation is known for example from Wikipedia (permanent link: https://de.wikipedia.org/w/index.php?title=Hermiteinterpolation&oldid=217101697).

Alternatively, other interpolations, for example a linear interpolation, could also be used, but this could produce a slightly poorer result.

In a further preferred embodiment of the arrangement according to the invention, a time stamp is assigned to each of the data points of the photoplethysmogram and the seismocardiogram, wherein the computer device is configured to synchronize the photoplethysmogram and seismocardiogram in time, in that the start time stamp of the later starting time curve of the photoplethysmogram or seismocardiogram is defined as the reference time and the data of the earlier starting time curve of the photoplethysmogram or seismocardiogram, which are before the reference time, are not taken into account. The time stamps are e.g. absolute time with nano-second resolution, as provided by Android smartphones for example. The time stamps can be added directly by the light sensor or the camera and the motion sensor when the measurement data is recorded or by the computer device immediately after receiving the measurement data from both sensors.

In other words, the reference time is defined as the starting point. If the reference time is then subtracted from both timelines, both timelines start at the time point to =0 s.

The synchronized time curves of photoplethysmogram and seismocardiogram may be inconsistent with regard to their sampling time points. It is therefore advisable to resample again to a common sampling rate or time resolution in order to obtain equidistant sampling time points on both time curves. Preferably, the Hermite interpolation can be used again to achieve a sampling rate of 1000 Hz for example.

In a further preferred embodiment of the arrangement according to the invention the computer device is configured to process the photoplethysmogram and/or the seismocardiogram with a band-pass filter to reduce interference. For example a band-pass filter can be used which has a lower threshold value for removing a slow moving average (“slow moving average removal filter”). An upper threshold value (“faster moving average noise removal filter”) can be used to remove interference (“noise”).

A series of trials have shown that a lower threshold of 0.5 Hz and an upper threshold of 30 Hz are particularly suitable for the seismocardiogram. For the seismocardiogram a lower threshold of 0.5 Hz and an upper threshold of 100 Hz are particularly suitable.

In a further preferred embodiment of the arrangement according to the invention, the computer device is configured to recognize local minima and local maxima in the photoplethysmogram by means of a sliding time window of predetermined duration and to assign each to pair of extreme values, and to determine a first pulse time point on the basis of a mean amplitude level for each pair of extreme values. The sliding time window has for example a length of 150 ms to 450 ms, preferably 200 ms to 400 ms, even more preferably of 250 ms to 350 ms. Ideally, the length of the sliding time window is adjusted to the duration of a heartbeat and lasts 300 ms for example. In other words, successive minima and maxima in the time window are assigned to each other to form a pair of extreme values, e.g. a local maximum following a local minimum. A mean amplitude level is characterized for example in that if a vertical auxiliary line is placed through the first pulse time point which intersects the two horizontal auxiliary lines through the minimum and das maximum, the section of the vertical auxiliary line lying above the mean amplitude level has the same length as the section lying below the mean amplitude level. In other words, the mean amplitude level characterizes half a wave height of the heartbeat.

This approach has the advantage of being particularly accurate and insensitive to interference in the PPG. For many people, the maximum and the minimum often have a roughly flat progression with numerous small fluctuations in the measurement, so that the exact time point of the local minimum and local maximum cannot be determined exactly. This inaccuracy can be reduced by using the mean amplitude level.

A mathematical method for using a sliding window to determine minima and maxima is known from the publication “STREAMING MAXIMUM-MINIMUM FILTER USING NO MORE THAN THREE COMPARISONS PER ELEMENT” by Daniel Lemire, Nordic Journal of Computing, Volume 13, Number 4, pages 328-339, 2006, and can be used advantageously in the context of the present invention.

In another preferred embodiment of the arrangement according to the invention, the computer device is configured to recognize local minima in the photoplethysmogram by means of a sliding time window of predetermined duration, and to determine a first pulse time point on the basis of the respective local minimum. This alternative configuration uses the local minimum instead of the mean amplitude level.

In a further preferred embodiment of the arrangement according to the invention, the computer device is configured to recognize local maxima in the seismocardiogram by means of a sliding time window of predetermined duration and to provide the time point of a local maximum as the second pulse time point. The local maxima correspond to the vibration triggered by the opening of the aortic valve.

The determination of the extreme values in connection with the second pulse time point is carried out using the same approach as explained above for the PPG. For example, a first or second derivation can be analyzed in each case.

In a further preferred embodiment of the arrangement according to the invention, the computer device is configured to determine the pulse wave transit time on the basis of a time difference between the first and the second pulse time point. In other words, the duration of the pulse beat is estimated in each case for the pulse time points assigned to each other, i.e. in the same sliding window, on the basis of the transit time of the pulse wave from the detection of the opening of the aortic valve in the SCG up to the arrival of the pulse beat, e.g. at the fingertip, recognizable in the PPG. Advantageously, if the measurement period comprises multiple heart beats, e.g. 60-80 heart beats per minute, a histogram of the pulse wave transit times determined for each heartbeat can be created and an average pulse wave transmit time can be determined using the median in relation to the histogram. Thus further increases the accuracy of the method.

Furthermore, alternatively or additionally, an average of all pulse wave transit times recorded during a measurement period (of e.g. one minute or 80 heartbeats) can be formed and a deviation measurement calculated. Pulse wave transit times, that deviate from the average by more a specified deviation measurement can be discarded as incorrect. If for example an empirical standard deviation is calculated, the specified deviation can be set to +/−3 sigma (this comprises 99.7% of all measured values).

The correctness of the measurement can be checked in each case by checking whether the second pulse time point in the SCG is always recognizable before the first pulse time point in the PPG, as the pulse wave must travel from the heart to the periphery. If this is not the case, the assignment according to the sliding time window may not be correct. The measurement should be repeated.

In a further preferred embodiment of the arrangement according to the invention, the computer device is configured to take into account a design-related time offset of the arrangement. An offset within the meaning of the invention is a time difference between two measured values. Surprisingly, it has been shown that under certain circumstances, when using different models of smartphones in the context of the arrangement according to the invention, significantly different pulse wave transit times are determined when the same person is tested. The offset refers to different pulse wave transit times.

To further improve the accuracy of the determination of the pulse wave transit time, a calibration measurement can therefore be carried out with several test subjects for a series of devices (smartphones, tablets, etc.). After this the detected pulse wave transit times determined for each person can then be compared with a reference value which is determined for example by an independent measurement using another method than the method according to the invention or by forming an average or median of the pulse wave transit times determined for the tested devices. In each case, an average deviation of the pulse wave transit time from the reference vale can be determined for each tested arrangement, which can be taken into account as an offset for all future measurements with the relevant model of smartphone or tablet. In this way, design-related deviations in measurement results between different models can be largely equalized. One useful application is for the manufacturer to provide a database with a time offset value for each arrangement. For example, when using a smartphone with an app, the app can read out the model of the smartphone and download the appropriate offset value to the device.

In a further preferred embodiment of the arrangement according to the invention the computer device is configured to take into consideration a design-related time offset of the arrangement, wherein the offset is determined by means of a pulse that is recognized simultaneously in the light signal and in the motion signal. The offset relates in this example to a difference in the time stamps for the motion signal and the light signal for events which are measured at the same time.

For example, a sound or a noise can be emitted as a sound pulse before the actual measurement, wherein the arrangement is for example a smartphone or tablet and is placed on a hard surface such as a table with the camera facing upwards. If the sound pulse is emitted by using a loudspeaker, the camera image will blur slightly if the sound is strong enough to cause the device to vibrate slightly. On the other hand, at the same moment the motion signal will allow the resonance to be recognized. Both events are easily recognizable using standard signal processing methods and the corresponding time stamps of the two signals, at which the pulse can be identified. The difference between these two time stamps is the offset, which is now used to better synchronize the two data streams of the motion signal and light signal. Typically, the offset can be between 100 ms to 200 ms.

Another approach is to use the vibration alarm of a smartphone or tablets as the pulse. The vibrations cause stronger movements of the device and when placed on a table generate rhythmic movements that are comparatively easy to recognize and also have a strong influence on the camera image. The use of the vibration alarm is therefore preferable to the use of a sound.

In a further preferred embodiment of the arrangement according to the invention the computer device is configured to take into account a design-related time offset of the arrangement, wherein the offset is determined by means of a sound pulse, which is simultaneously recognized as a sound signal by means of a microphone and in the motion signal. The sound pulse can be tone or a noise emitted by a loudspeaker, or it can be a vibration alarm.

This further approach is characterized by the fact that smartphones and tablets often already have very good time synchronization between a microphone and the camera, in order to synchronously capture the image and sound during video recordings. This method can be used in a simple and uncomplicated way, whereby it is not necessary to analyze the light signal or a video recording.

Although the use of a microphone is disclosed in the aforementioned publication WO 2014/072461, this is only used to take into account the sound of a pulse wave travelling through the captured image as additional information.

The problem of the design-related time offset can be avoided if an arrangement is designed specifically for use in determining the pulse wave transit time. In this case, the design-related offset, if it occurs between the two sensors, can be determined once by the manufacturer and taken into account for each subsequent measurement. Typically in many countries, such a newly developed device requires a more extensive medical device license than is necessary when using conventional hardware in the form of smartphones or tablets.

Based on previous approaches for determining pulse wave transit times, the invention also has the task of specifying a computer-implemented method, by which a pulse wave transit time can be determined comparatively simply, accurately and inexpensively.

The invention solves this problem by means of a computer-implemented method according to claim 11. Preferred embodiments of the computer-implemented method are given in the dependent claims 12 to 14. The same advantages as described at the beginning for the arrangement according to the inventive are achieved analogously. The person skilled in the art can easily extend the present teaching for computer-implemented methods by the embodiments explained in connection with the arrangement.

The computer-implemented method can for example be provided as software in the form of an app on the Apple iOS or Android operating system. The same applies to the computer program product described in the following.

Based on previous approaches for determining pulse wave transit times, the invention also has the task of specifying a computer program product with which a pulse wave transit/propagation time can be determined comparatively simply, accurately and inexpensively.

The invention solves this problem by means of a computer program product according to claim 15. This has the same advantages as described above for the arrangement and the computer-implemented method according to the invention. The person skilled in the art can easily extend the present teaching for the computer program product by the embodiments explained in connection with the arrangement.

To provide a better explanation of the invention, preferred exemplary embodiments of the invention are explained in the following with reference to the drawings. The various exemplary embodiments as well as the embodiments of the preceding description can be combined with one another freely in order to specify the solution according to the invention in a wide variety of ways.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In a schematic representation:

FIG. 1 shows a first exemplary embodiment of an arrangement according to the invention, and

FIG. 2 shows an exemplary embodiment of a computer-implemented method according to the invention, and

FIG. 3 shows an exemplary embodiment of a computer program product according to the invention, and

FIG. 4 shows a second exemplary embodiment of an arrangement according to the invention, and

FIG. 5 shows a third exemplary embodiment of an arrangement according to the invention, and

FIG. 6 shows a fourth exemplary embodiment of an arrangement according to the invention, and

FIG. 7 shows a representation of suitable surfaces for a measurement of a pulse wave transit time, and

FIG. 8 shows a representation of a suitable positioning of an arrangement according to the invention, and

FIG. 9 shows an exemplary embodiment for a synchronization and resampling of a photoplethysmogram and a seismocardiogram, and

FIG. 10 shows an exemplary embodiment for determining a first pulse time point, and

FIG. 11 shows a photoplethysmogram and a seismocardiogram with a determination of the pulse wave transit time.

### DETAILED DESCRIPTION

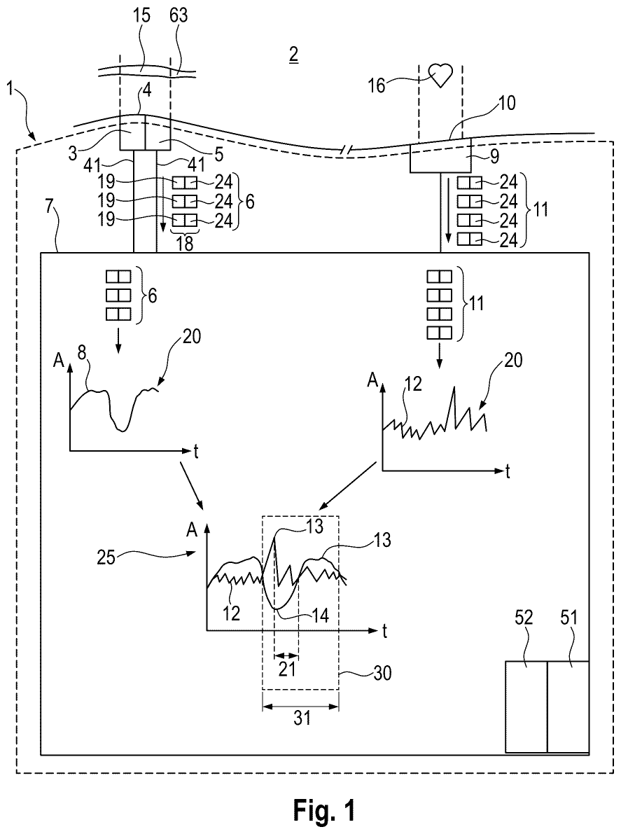

FIG. 1 shows an arrangement 1 for determining a pulse wave transit time 21 of an object 2 comprising a light source 3, which is configured to irradiate a first surface 4 of the object 2 with light. A light sensor 5 is configured to detect light emanating from the first surface 4—e.g. an area of skin on the fingertip of a human or animal body or the object 2 and provide it as a light signal 6. An area 15 of the object 2, in which an arterial blood vessel 63 is located, is assigned to the first surface 4.

In the example, the light signal has a timeline 18 of individual images 19, wherein each individual image 19 is provided with a time stamp 24 which indicates the time point of the data acquisition. This timeline is transmitted to a computer device 7 via a data communication link 41.

The computer device 7 is configured to calculate a photoplethysmogram 8 on the basis of the light signal 6. This is a time curve of color change values, the amplitude A of which is plotted against the time t as a time curve 20.

A motion sensor 9 is arranged on a second surface 10 of the object 2, wherein an area 16 of the object 2, in which the heart is located, is assigned to the second surface 10. The motion sensor 9 detects a movement of the second surface 10 as a motion signal 11. This consists of acceleration values in several spatial dimensions, wherein a time stamp 24 is assigned to each acceleration value or data point 23. The computer device 7 uses the motion signal 11 to provide a seismocardiogram 12.

The computer device performs a time synchronization 25 of the photoplethysmogram 8 and seismocardiogram 12. Local extreme values 13, 14 are determined in the respective time curve 20 of the photoplethysmogram 8 and seismocardiogram 12. The pulse wave transit time 21 is then determined, taking into consideration the local extreme values 13, 14. A sliding time window 30 of predetermined duration 31 is used for this.

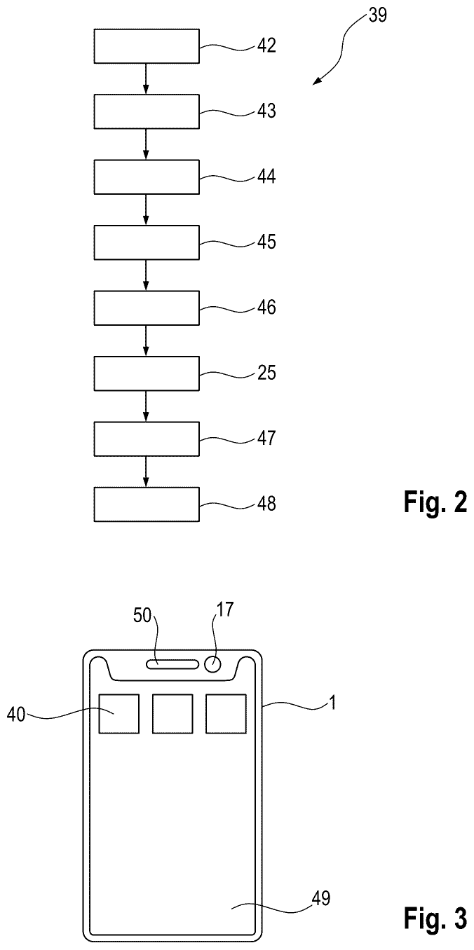

The FIG. 2 shows a computer-implemented method 39 for determining a pulse wave transit time of an object, comprising the steps:
irradiating (42) a first surface of the object with light by means of a light source, and providing (43) of a light signal, which is detected by means of a light sensor as light emanating from the first surface of the object, and
calculating (44), by means of a computer device, a photoplethysmogram on the basis of the light signal, and the steps:
detecting (45) a movement of a second surface of the object as a motion signal by means of a motion sensor, and
providing (46), by means of the computer device, a seismocardiogram on the basis of the motion signal, and
synchronizing in time (25) the photoplethysmogram and the seismocardiogram, and
determining (47) in each case local extreme values in the time curve of the photoplethysmogram and the seismocardiogram, and
determining (48) the pulse wave transit time between an area of the object associated with the first surface and an area of the object associated with the second surface of the object, taking into consideration the local extreme values.

FIG. 3 shows an arrangement 1, which is equipped as a smartphone with a front camera 17 as a light sensor and a loudspeaker 50. A touch-sensitive screen (“touchscreen”) is provided as a display means 49. Various executable programs are displayed on the screen as so-called apps, which can be selected and activated by a user by touching the screen. Each app represents a computer program product. The computer program product 40 is configured to carry out the method according to the invention using the hardware and firmware of the smartphone and an operating system such as e.g. Android or Apple iOS.

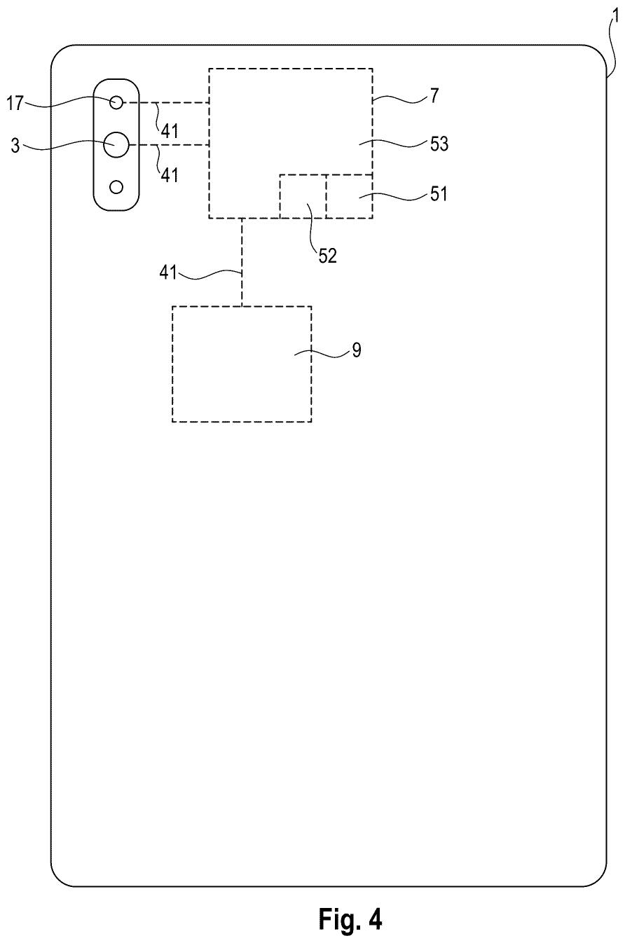

FIG. 4 shows an arrangement 1, which is configured as a smartphone. The device is shown in a view of the rear side facing away from the screen. A camera 17 (the light sensor) is in the form of a color camera.

Furthermore, the flash of the device can be used as a light source 3. The light source 3 and camera 17 are connected via data communication links 41 to a computer device 7, which comprises a microprocessor 53 with a data storage device 51 and a working memory 52. A motion sensor 9 is connected to the computer device 7 via a data communication link 41. The components 7, 9, 41, 51, 52 represented by dashed lines are not visible from the outside on the back of the smartphone.

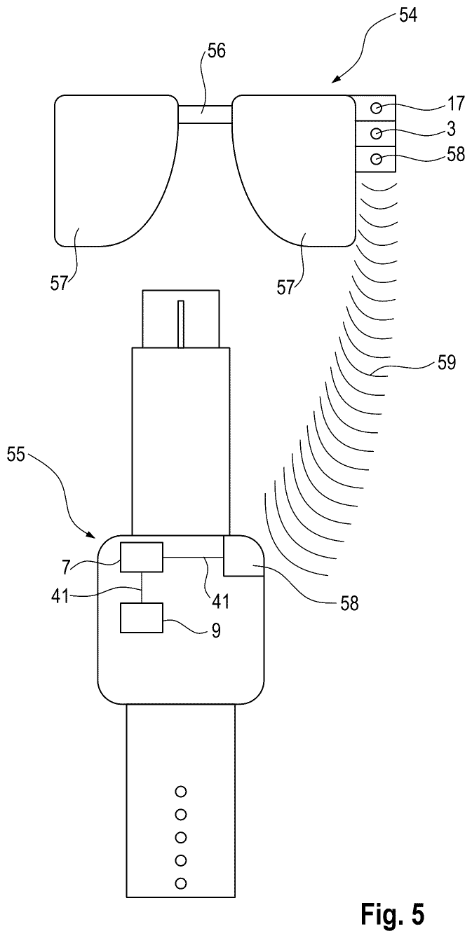

FIG. 5 shows another embodiment of the arrangement 1 according to the invention, in which smart glasses 54 and smart glasses and smartwatch 55 (i.e. glasses with a display device for data) are used. The smart glasses 54 have two lenses 57, which are also used for displaying data. The data can be displayed for example by pixels incorporated into the lenses or by a projector, preferably by means of micro-electromechanical systems (MEMS). MEMS are microscopically small controllable mirrors that can be used to project images onto the inside of the lenses. MEMS are generally manufactured as semiconductor components made of silicon. Alternatively, the lenses can be simply screens in the form of data glasses. Here the glasses are configured to be opaque for example, but can display images from a camera so that users can orient themselves in their surroundings.

The lenses 57 are connected by a bridge 56. A flash is used as a light source 3, a camera 17 is used as a light sensor. A light signal can be transmitted to a smartwatch 55 by radio communication device 58 via radio data transmission 59 and received there. The smartwatch 55 comprises a microprocessor as a computer device 7. A motion sensor 9 is provided in the smartwatch 55 so that a motion signal can be detected in the smartwatch 55.

In this exemplary embodiment the user can position the smartwatch 55 on their sternum and cover the camera 17 and the flash with a fingertip. In this way, the series of measurements for determining the pulse wave transit time can be carried out.

Many different data communication standards can be used for the radio transmission, such as e.g. 2G, 3G, 4G, 5G, W-LAN, Bluetooth, NFC. Preference is given to data communication standards which like some Bluetooth variants already have mechanisms for the time synchronization of devices via radio data transmission 59.

In a further development of this embodiment it is possible to omit the other respective device of the smart glasses and smartwatch device pair, if all the components required for the pulse wave transit time calculation are installed in one device. This also eliminates the radio data transmission connection. For example, smart glasses can be equipped with a flash, camera, motion sensor and microprocessor. The user then places the glasses on the sternum, as explained above for the smartphone, and covers the flash and camera with a fingertip. Alternatively, all of the aforementioned components can also be integrated into a smartwatch. It is understood that the smart glasses or smartwatch can also be coupled, together or separately, to other receiving devices, such as a tablet, smartphone or other data processing devices, such as a laptop or a stationary PC.

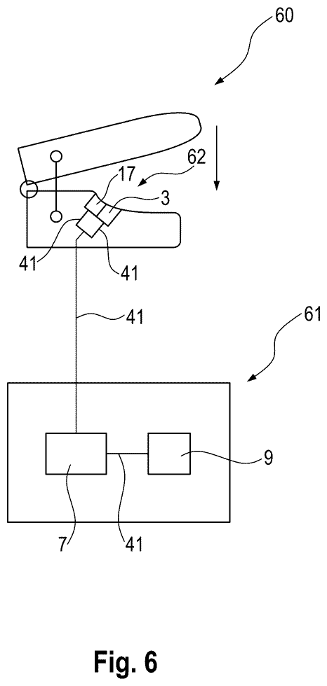

FIG. 6 shows an arrangement, in which application-specific devices have been constructed. A finger measuring device 60 comprises a receiving area 62 for a finger (not shown), which is equipped with a light source 3 and a camera 17. The finger measuring device 60 fits like a clamp on the finger in the manner of a pulse oximeter (see arrow). A vibration measuring device 61 is equipped with a motion sensor 9 and a computer device 7.

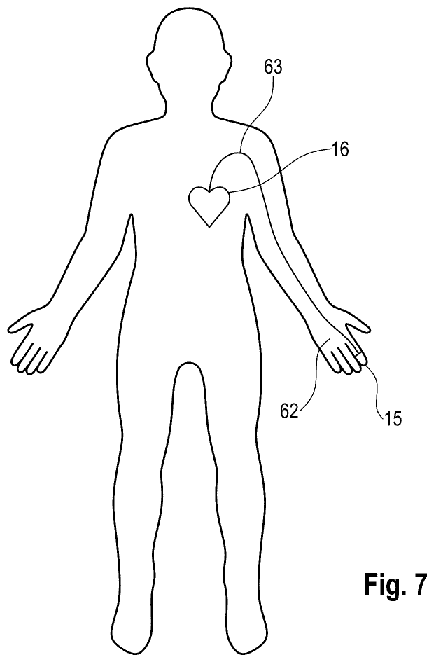

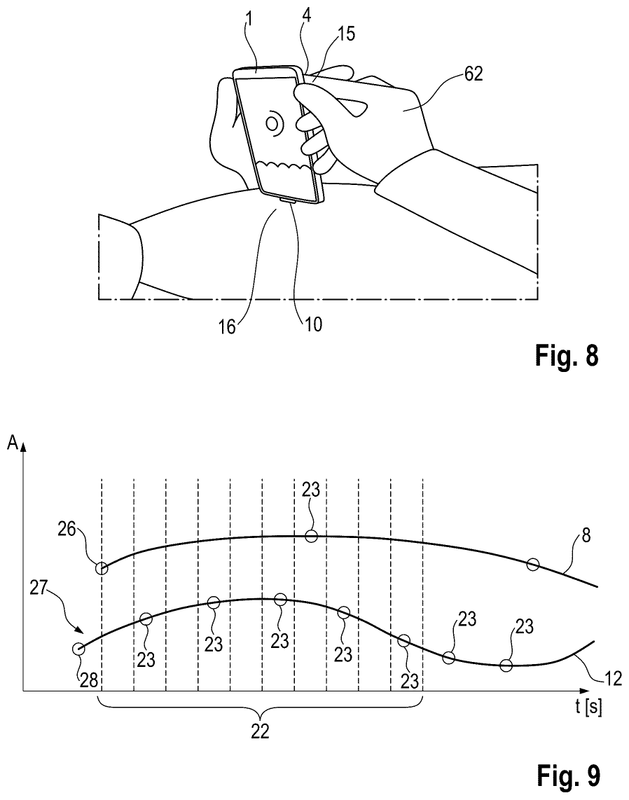

FIGS. 7 and 8 show a suitable positioning for an arrangement 1, in this case a smartphone, for measuring the pulse wave transit time. The dominant hand 62 of the user lies with the fingertip as the first surface 4, subcutaneous tissue of which with an arterial blood vessel 63 forms the area 15 assigned to the first surface 4, on the rear camera (i.e. the side facing away from the screen) and the flash of the smartphone. The sternum forms the second surface 10 with the associated area 16, the user's heart.

FIG. 9 shows an example of a synchronization and resampling (to a common sampling rate) of a photoplethysmogram 8 and seismocardiogram 12. The photoplethysmogram 8 has only three data points 23, 26, while the seismocardiogram 12 has eight data points 23, 28 in the same time period. The data points can each be arranged on the time axis using the time stamps attached to the original light signal 6 and the original motion signal 8. The seismocardiogram 12 therefore has a much higher resolution than the photoplethysmogram 8.

In the example shown, the first data point 28 of the seismocardiogram 12 is arranged before the first data point 23 of the photoplethysmogram 8 in time. In order to synchronize both evaluations using the time stamps, the first data point of the photoplethysmogram 8 is defined as the reference time point or start time stamp 26 of both timelines. Both timeline 8, 12 are brought to a higher common resolution of e.g. 1000 Hz by means of Hermite interpolation, i.e. upsampling is performed. In this case the resampling 22 is therefore an upsampling of the data points 23,26 (this results in a plurality of new data points, indicated by the intersections of the dashed lines with the seismocardiogram 12 and the photoplethysmogram 8). The data points 28 located before the start time stamp 26 are not taken into account in the further evaluation.

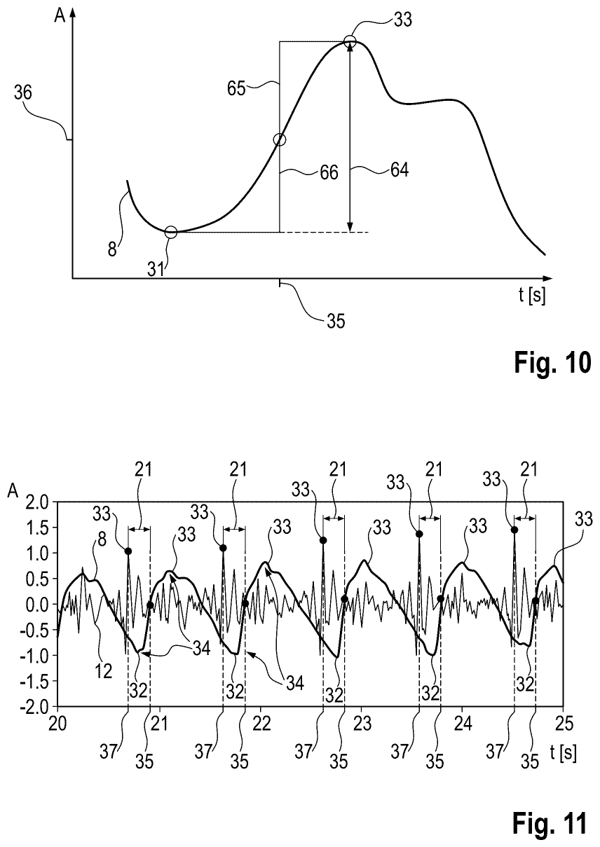

FIG. 10 shows an example for determining a first pulse time point 35 in the photoplethysmogram 8. Here the mean amplitude level 36 is defined as a point in the time curve of the photoplethysmogram 8, at which the upper segment 65 of the maximum amplitude 64 is the same length as the lower segment 66 of the maximum amplitude 64, i.e. both segments 65, 66 have half the length of the maximum amplitude 64. The maximum amplitude 64 is the distance between the minimum 31 and the maximum 33. On the time axis, the first pulse time point 35 results as soon as the mean amplitude level 36 has been found.

FIG. 11 shows a photoplethysmogram 8 and a seismocardiogram 12, wherein the amplitude A of the respective timeline has been plotted against the time t in seconds. In each case minima 32 and maxima 33 have been identified, wherein pairs of extreme value 34 have been identified for the photoplethysmogram 8 (these were assigned using the sliding time window as explained above). The maxima 33 of the seismocardiogram 12 each mark the second pulse time points 37, which occur per heartbeat before the first pulse time points 35 of the photoplethysmogram 8, wherein the second pulse time points 37 are located at the time points of the mean amplitude level between the respective minimum and the respective maximum of the pulse wave. The pulse wave transit time 21 results in each case as a difference between the two pulse time points 35, 37.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

The meaning of the reference signs used in the drawings is given in the following list of reference signs. In the drawings, the same reference signs always denote the same component or method steps.

Reference Signs

| | |
|---|---|
| 1 | arrangement |
| 2 | object |
| 3 | light source |
| 4 | first surface |
| 5 | light sensor |
| 6 | light signal |
| 7 | computer device |
| 8 | photoplethysmogram |
| 9 | motion sensor |
| 10 | second surface |
| 11 | motion signal |
| 12 | seismocardiogram |
| 13, 14 | local extreme values |
| 15 | area assigned to the first surface |
| 16 | area assigned to the second surface |
| 17 | camera |
| 18 | timeline of individual images |
| 19 | individual images |
| 20 | one-dimensional temporal progression |
| 21 | pulse wave transit time |
| 22 | resampling |
| 23 | data points |
| 24 | time stamp |
| 25 | synchronization |
| 26 | start time stamp |
| 27 | earlier starting time curve |
| 1 | arrangement |
| 28 | early data points |
| 29 | band-pass filter |
| 30 | sliding time window |
| 31 | predefined duration |
| 32 | local minima |
| 33 | local maxima |
| 34 | pair of extreme values |
| 35 | first pulse time point |
| 36 | mean amplitude level |
| 37 | second pulse time point |
| 38 | time difference |
| 39 | computer-implemented method |
| 40 | computer program product |
| 41 | data communication link |
| 42 | irradiation |
| 43 | provision of a light signal |
| 44 | calculation of a photoplethysmogram |
| 45 | detection of a movement |
| 46 | provision of a seismocardiogram |
| 47 | determination of respective local extreme values |
| 48 | determination of pulse wave transit time |
| 49 | screen |
| 50 | loudspeaker |
| 51 | data storage device |
| 52 | working memory |
| 53 | microprocessor |
| 54 | smart glasses |
| 55 | smartwatch |
| 56 | bridge of glasses |
| 57 | lens |
| 1 | arrangement |
| 58 | radio communication device |
| 59 | radio data transmission |
| 60 | finger measuring device |
| 61 | vibration measuring device |
| 62 | dominant hand |
| 63 | arterial blood vessel |
| 64 | maximum amplitude level |
| 65 | upper segment of maximum amplitude |
| 66 | lower segment of maximum amplitude |

1. An arrangement for determining a pulse wave transit time of an object, comprising
a light source, which is configured to irradiate a first surface of the object with light, and
a light sensor, which is configured to detect light emanating from the first surface of the object and to provide as a light signal, and
a computer device, which is configured to calculate a photoplethysmogram-on the basis of the light signal,
wherein
a motion sensor is configured to detect a movement of a second surface of the object as a motion signal, and in that
the computer device is configured to provide a seismocardiogram on the basis of the motion signal and
to synchronize in time the photoplethysmogram and the seismocardiogram, and
to determine local extreme values over the course of time of the photoplethysmogram and the seismocardiogram, and
taking into consideration the local extreme values, to determine the pulse wave transit time between an area of the object associated with the first surface and an area associated with the second surface of the object.

2. The arrangement according to claim 1, wherein the light sensor comprises a camera which is configured to capture a timeline of individual images with a predetermined frame rate and predetermined image resolution.

3. The arrangement according to claim 2, wherein the computer device is configured to convert the individual images into a one-dimensional time curve, wherein for each individual image color values of pixels are added up, and to provide these added up color values in the time curve as a photoplethysmogram.

4. The arrangement according to claim 1, wherein the computer device is configured to carry out a resampling (22) of the photoplethysmogram (8) and the seismocardiogram (12) to a common time resolution.

5. The arrangement according to claim 4, wherein the computer device is configured to use a Hermite interpolation for the resampling.

6. The arrangement according to claim 1, wherein time stamps are assigned respectively to data points of the photoplethysmogram and the seismocardiogram, and in that the computer device is configured,
to synchronize in time the photoplethysmogram and the seismocardiogram, in that the start time stamp of the later starting time curve of the photoplethysmogram or seismocardiogram is defined as a reference time point and early data points of the earlier starting time curve of the photoplethysmogram or seismocardiogram, which are before the reference time, are not taken into account.

7. The arrangement according to claim 1, wherein the computer device is configured, to process the photoplethysmogram and/or the seismocardiogram with a band-pass filter in order to reduce interference.

8. The arrangement according to claim 1, wherein the computer device is configured, to detect local minima and local maxima in the photoplethysmogram by means of a sliding time window of predetermined duration and assign to each a pair of extreme values, and to determine a first pulse time point on the basis of a mean amplitude level for each pair of extreme values.

9. The arrangement according to claim 8, wherein the computer device is configured to detect local maxima in the seismocardiogram by means of the sliding time window of predetermined duration and to provide the time point of a local maximum in each case as a second pulse time point.

10. The arrangement according to claim 9, wherein the computer device is configured to determine the pulse wave transit time in each case on the basis of a time difference between the first and the second pulse time point.

11. A computer-implemented method for determining a pulse wave transit time of an object, comprising the steps:
irradiating a first surface of the object with light by means of a light source, and
providing a light signal, which is detected by means of a light sensor as light emanating from the first surface of the object, and
calculating, by means of a computer device, a photoplethysmogram on the basis of the light signal,
wherein the steps:
detecting a movement of a second surface of the object as a motion signal by means of a motion sensor, and
providing, by means of the computer device, a seismocardiogram on the basis of the motion signal, and
synchronizing in time the photoplethysmogram and the seismocardiogram, and
determining in each case local extreme values in the time curve of the photoplethysmogram and the seismocardiogram, and
determining the pulse wave transit time between an area of the object associated with the first surface and an area of the object associated with the second surface, taking into consideration the local extreme values.

12. The computer-implemented method according to claim 11, wherein local minima and local maxima are detected by means of the computer device in the photoplethysmogram by means of a sliding time window of predetermined duration and are assigned respectively to a pair of extreme values, and in that
a first pulse time point is determined for each pair of extreme values on the basis of a mean amplitude level.

13. The computer-implemented method according to claim 12, wherein local maxima are detected by means of the computer device in the seismocardiogram by means of the sliding time window of predetermined duration and in each case the time point of a local maximum is provided as a second pulse time point.

14. The computer-implemented method according to claim 13, wherein the pulse wave transit time is determined by means of the computer device in each case on the basis of a time difference between the first and the second pulse time point.

15. A computer program product, comprising commands, which when the program is executed by a computer, cause the computer to execute the computer-implemented method according to claim 11.
